(19) United States
(12) Patent Application Publication
(10) Pub. No.: US 2006/0115522 A1
(43) Pub. Date: Jun. 1, 2006

(54) Pharmaceutical composition

(75) Inventors: Amar Lulla, Maharashtra (IN); Geena Malhotra, Maharashtra (IN)

Correspondence Address: STEVENS DAVIS MILLER & MOSHER, LLP, 1615 L STREET, NW, SUITE 850, WASHINGTON, DC, 20036, US

(73) Assignee: CIPLA LTD, Mumbai Central (IN)

(21) Appl. No.: 10/545,004

(22) PCT Filed: Feb. 17, 2004
(86) PCT No.: PCT/GB04/00584

§ 371 Date: Oct. 31, 2005

(30) Foreign Application Priority Data
Feb. 17, 2003 (IN) 194/MUM/2003

(51) Int. Cl. A61K 38/13; A61K 31/4745; A61F 13/02; A61L 15/16

(52) U.S. Cl. 424/448; 514/11; 514/291; 514/183

(57) ABSTRACT

A patch, or patch forming composition, comprising at least one immunosuppressant, or a pharmaceutically acceptable salt, solvate or physiologically functional derivative thereof, together with a pharmaceutically acceptable carrier or excipient therefor, formulated for delivery of said least one immunosuppressant, or a pharmaceutically acceptable salt, solvate or physiologically functional derivative thereof to the skin of a patient, and methods of treatment employing the same.

The present invention is concerned with topical immunotherapy and compositions suitable for use therein.

Topical immunotherapy can be used to describe topical treatment with an agent having immunomodulatory properties. Recently, topical formulations including agents with direct immunosuppressive actions have been tested in diseases believed to have an immunological basis, especially atopic dermatitis and psoriasis. These topical immunosuppressive agents have included tacrolimus, asomycin and cyclosporin and their derivatives, and other structurally related macrolide topical immunosuppressants.

Tacrolimus is a hydrophobic macrolide immunosuppressant produced by Streptomyces tsukubaensis No. 9993. Tacrolimus, 17-allyl-1,14-dihydroxy-12-[2-(4-hydroxy-3-methoxycyclohexyl)-1-methylvinyl]-23,25-dimethoxy-13,1 9,21,27-tetramethyl-11,28-dioxa-4-azatricyclo[22.3.1.0^{4.9}]octacos-18-ene-2,3, 10,16-tetraone, which is also known as FK-506 or FR-900506, has the following structural formula:

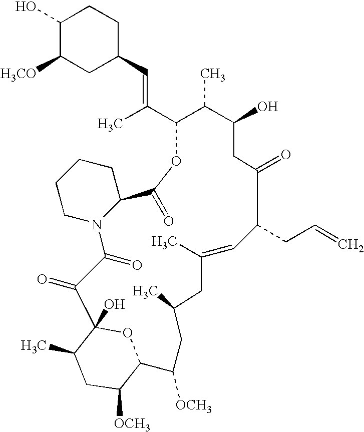

Tacrolimus inhibits T-lymphocyte activation by first binding to an intracellular protein, FKBP-12. A complex of tacrolimus-FKBP-12, calcium, cadmodulin and calcineurin is then formed and the phosphatase activity of calcineurin is inhibited. The effect has been shown to prevent the dephosphorylation and translocation of nuclear factor of activated T-cells (NF-AT), a nuclear component thought to initiate gene transcription for the formation of lymphokines (such as interleukin-2, gamma interferon). Tacrolimus also inhibits the transcription for genes which encode IL-3, IL4, IL-5, GM-CSF and TNF-alpha, all of which are involved in the early stages of T-cell activation. Tacrolimus inhibits proliferation and selective cytokine expression in antigen stimulated T cells in culture and also inhibits B cell proliferation at similar concentrations.

More particularly, tacrolimus inhibits T-lymphocyte activation, having a direct effect on T-lymphocytes so as to inhibit IL-2 transcription, which decreases responsiveness of T-lymphocytes to foreign antigens. The action of tacrolimus on atopic dermatitis may be related to alteration of antigen presenting cells, suppression of IL-2 and co-stimulatory molecule expression, impairment of phenotypic and functional differentiation of epidermal Langerhans' cells and suppression of Th1 and Th2 cytokine induction in lymph node cells. The effect of tacrolimus on pruritis may be related to inhibition of histamine release from skin mast cells and impairment of de novo mast cell prostaglandin D2 synthesis along with diminished release of histamine from basophiles. It has also been reported that in atopic dermatitis patients, tacrolimus does not alter collagen synthesis and it not atrophogenic.

Tacrolimus is available in both intravenous and oral formulation for the prevention of organ rejection after allogeneic liver or kidney transplantation. Oral tacrolimus has been found to be useful in the treatment of psoriasis, but potentially serious side effects, such as nephrotoxicity and hypertension, has limited its use for dermatologic indications by this route of administration. Topical formulations (ointments) have been extensively studied and reported to show positive effects in treatment of inflammatory skin diseases, such as atopic dermatitis and psoriasis. Tacrolimus administered topically (as an ointment) has been reported to be safe and effective in the treatment of skin diseases.

Ascomycin is the fermentation product of Streptomyces hygroscopicus var. ascomyticus. It includes SDZASM 981, ABT-281 and SDZ-240. These are effective against psoriasis, allergic contact dermatitis and atopic dermatitis, and their mechanism of action is similar to tacrolimus.

Cyclosporin is a neutral cyclic peptide composed of 11 amino acids. It acts by inhibiting interleukin-2 (IL-2) production by activated CD4+ T cells. Impaired IL-2 production leads to a decline in the number of activated CD4 and CD8 cells in the epidermis. It is most effective against psoriasis, but also has efficacy against atopic dermatitis, lichen planus, pemphigus, epidermolysis bullosa acquisata, dermatomyositis, sclerodema, pyoderma gangrenosa, contact dermatitis and other dermatological disorders.

The following prior art patent documents describe known tacrolimus formulations—US 2002173516, US 2002052407, US 2002013340, EP 1092429, U.S. Pat. No. 6,187,756, U.S. Pat. No. 6,184,248, EP 1067926, HU 0000587 and WO 98/09523.

US 2002052407, U.S. Pat. No. 6,187,756, U.S. Pat. No. 6,184,248 and WO 98/09523 describe compositions of tacrolimus for use in neurological disorders and neurogenerative diseases.

EP 1092429 discusses pharmaceutical compositions and methods for treating immune response associated disorders.

US 2002173516 and 2002013340 describe pharmaceutical compositions and methods for treating immune response associated diseases of the surface and anterior segment of the eye.

EP 1067926 and WO 99/51215 describe the use of tacrolimus as showing inhibitory activity on the production of nitric oxide.

WO 02/092031, U.S. Pat. No. 6,492,427 and EP 0547229 disclose topical formulations that employ cyclosporin for the treatment of alopecia, psoriasis, atopic dermatitis, multiple sclerosis and other dermatological disorders.

U.S. Pat. No. 5,925,649 discloses preparation of ascomycin and a topical formulation thereof to be used as creams, lotion, emulsion, transdermal patch, cataplasm, plaster and gels. WO 00/32234 discloses an ointment of ascomycin along with a hydrocarbon and a fatty alcohol or a fatty oil.

Transdermal systemic drug delivery provides an effective method of achieving improved bioavailability for physiologically active substances. Transdermal drug formulations stabilized with polymers form an appreciable surface mass on the skin, which can be retained on the skin over a prolonged duration of many hours. Transdermal delivery of such drugs can also be achieved by a metered dose transdermal spray so that a fixed amount of drug can be delivered per actuation, thus maintaining dose therapeutics.

U.S. Pat. No. 6,455,066 discloses a patch comprising a backing and a pressure sensitive acrylic adhesive, the adhesive comprising a therapeutically effective amount of lidocaine, or a pharmaceutically acceptable salt thereof, and a penetration enhancing amount of soybean oil for administration of anesthesia.

U.S. Pat. No. 5,540,931 discloses a formulation for treatment of skin proliferative disorders by topical administration of an immunosuppressant, such as tacrolimus, in a suitable carrier, such as an oily vehicle. Although a patch is referred to as an alternative transdermal delivery mode, no specific enabling disclosure of such a patch for an immunosuppressant is provided.

U.S. Pat. No. 6,190,691 and U.S. Pat. No. 6,224,901 respectively describe formulations comprising cyclosporin as an anti-inflammatory agent and to increase the growth of hair follicles for treating alopecia.

Although it is well known in the prior art that tacrolimus, ascomycin and cyclosporins have been used for topical immune therapy, there has been a need for an improved dosage form that could enable the transfer of drugs in a more effective manner. Other topical dosage forms, such as emulsions, ointments, creams and lotions, which are specifically described and as such enabled by the prior art, require direct application to the skin surface and a prolonged contact time for therapeutic efficacy, thus resulting in decreased bioavailability and patient compliance. Furthermore, these dosage forms can also be sticky and oily in nature, thus discouraging reuse thereof by a patient.

There is, however, provided by the present invention a patch, or patch forming composition, comprising at least one imnmunosuppressant, or a pharmaceutically acceptable salt, solvate or physiologically functional derivative thereof, together with a pharmaceutically acceptable carrier or excipient therefor, formulated for delivery of said at least one immunosuppressant, or a pharmaceutically acceptable salt, solvate or physiologically functional derivative thereof, to the skin of a patient.

The use of such a patch, or patch forming composition, as provided by the present invention can be advantageous in alleviating many of the above described problems hitherto associated with topical formulations as provided in the prior art. For example, the application time for a patch is considerably reduced compared to other topical compositions and also penetration of the drug molecule through the skin is faster than with other conventional topical dosage forms. The transdermal route of delivery, and thus the faster rate of drug transfer, increases the bioavailability of the drug. Therefore, the shorter contact time, ease of application and better therapeutic efficacy, can result in increased patient compliance.

A patch, or patch forming composition, for use according to the present invention can comprise a spray-on patch, a transdermal patch, a physical patch or a controlled release patch. In the case where a spray-on patch is provided by the present invention, this can be advantageous in that the at least one imnmunosuppressant, or a pharmaceutically acceptable salt, solvate or physiologically functional derivative thereof, to be delivered thereby may be administered in a metered dose.

An immunosuppressant suitable for use in a patch, or patch forming composition, according to the present invention, is preferably selected from the group consisting of tacrolimus, cyclosporin, ascomycin, or a pharmaceutically acceptable salt, solvate or physiologically functional derivative thereof.

The term “physiologically functional derivative” as used herein denotes a chemical derivative of an immunosuppressant as described herein having the same or similar physiological function as the free base immunosuppressant and, for example, being convertible in the body thereto.

The immunosuppressant active ingredient is typically included in a patch, or patch forming composition, according to the present invention at a concentration in the range of 0.1 wt % to 5 wt % of the total patch composition.

Suitably a patch, or patch forming composition, according to the present invention may include a combination of pharmaceutically acceptable polymers present in the composition so as to modulate delivery to the skin of the at least one immunosuppressant substantially as hereinbefore described. Typically the combination of polymers can achieve substantially controlled delivery of the at least one immunosuppressant.

Preferably a patch, or patch forming composition, according to the present invention further comprises one or more of the following: at least one film forming agent, at least one film plasticiser and at least one organic solvent.

The film forming agent suitable for use in a patch, or patch forming composition, according to the present invention can include one or more of the following: poloxamers, acrylic acid and its derivatives, polyacrylic acid and its derivatives such as polybutylmethacrylate, polymethacrylic acid and polymethacrylate, ascorbyl palmitate, carbomer, carnauba wax, cellulose derivatives such as cellulose acetate phthalates, crosscarmellose sodium, hydroxyethyl cellulose, hydroxypropyl cellulose, hydroxypropyl methylcellulose, ethyl cellulose and related compounds, hydroxypropyl methyl cellulose phthalate, hypromellose phthalate, crosspovidone, polyvinyl acetate, cellulose nitrates, polyvinyl alcohol and derivatives and related compounds, cetyl alcohol and derivatives, microcrystalline wax, polyethylene glycol, polyurethane, polyvinyl acetate phthalate, povidone, silicone rubber and derivatives, shellac and triglyceride derivatives. These film forming agents are organic solvent soluble, and in particular are preferably soluble in organic solvents which are dermatologically compatible solvents, in other words solvents which are recognized as suitable for use in dermatological and pharmaceutical application.

In certain embodiments of the present invention it is possible to include a plurality of film forming agents in a patch composition of the invention, which combination of film forming agents when applied to the skin will form a flexible skin patch.

Film plasticisers can be included in a patch, or patch forming composition, according to the present invention so as to soften a polymer film formed by the above described film forming agents and to ensure that a resulting film is sufficiently flexible so that it can be applied onto the skin without cracking and peeling at least during the intended lifespan of a skin patch as provided by the present invention. Examples of suitable film plasticisers include polybutylphthalate, benzyl benzoate, dibutyl sebacate, dimethyl phthalate, dibutyl phthalate, triacetin, glycol and derivatives thereof, benzyl benzoate, glycerin, mineral oil, lanolin alcohols (such as C₁₋₈ alcohols), petroleum, polyethylene glycol, sorbitol, triethylene citrate, propylene glycol, chlorbutanol, castor oil and gelatin.

Organic solvents, in particular volatile organic solvents, can be included in a patch, or patch forming composition, according to the present invention so as to aid application to the skin by spraying. By way of example, one or more solvents may be selected from acetone, ethyl acetate and isopropanol. These solvents are preferred as they may additionally offer some bactericide activity. Other solvents that may suitably be employed can include one or more of the following: alcohols, for example C₁₋₁₀ alcohols, such as benzyl alcohol, ethanol, methanol, butanol, isobutanol, and diacetone alcohol, chlorinated hydrocarbons such as methylene chloride, carbon tetrachloride, trichloroethylene and chlorothene SM, esters such as methyl acetate, n-propyl acetate, n-butyl acetate, isobutyl acetate, amyl acetate, 2-ethyl hexyl acetate, duPont DBE and Exxate 500, 700, 900, glycol and ether/ester derivatives, ethylene glycol, PM acetate, butyl celluosolve, carbritol acetate, butyl carbritol acetate, hydrocarbons such as toluene and xylene, ketones such as methyl ethyl ketone and methyl butyl ketone, ethers such as dimethyl ether, benzyl benzoate, isopropyl myristate, acetonitrile, ethyl oleate, glycerol, glycofurol (tetraglycol), propylene glycol, polyethylene glycol (PEG), hexane, n-hexane, methylene chloride, methyl chloride, octyl dodecanol, trichloromethane, diethyl phthalate, and dibutyl phthalate. These solvents are volatile and are included in dermatological preparations at concentrations which do not cause substantial irritation to the skin and as such are pharmaceutically acceptable. Such solvents, on application to the skin, rapidly volatilize.

A patch, or patch forming composition, according to the present invention can further comprise one or more preservatives selected from celluloses, such as HPMC, HPC, methyl cellulose, ethyl cellulose and the like, alcohols selected from poly-vinyl alcohols, ethanol and the like, spans and tweens, polysorbates and other preservatives well known in the state of the art.

A patch as provided by the present invention may further comprise gauze on which the patch composition can be embedded or applied, together with an adhesive, suitably a pressure sensitive adhesive, for the purpose of fixing the gauze to the skin of a patient. Suitably the gauze can comprise one or more polymeric materials, such as polyurethane films and foams, polyethylene oxide and polyvinyl pyrrolidone hydrogels, hydrocolloids, calcium alginates and the like. The gauze may be opaque or transparent, varying in thickness, and may in certain preferred embodiments be waterproof.

Suitably a patch, or patch forming composition, according to the present invention may be formulated for use in the treatment of dermatophytosis and related disorders. Dermatophytoses are infectious diseases caused by a group of keratinophilic, parasitic fungi known as “dermatophytes”. Dermatophyte infection can affect various keratinous tissues, such as the hair and stratum comeum of the skin.

Alternatively, a patch, or patch forming composition, according to the present invention is formulated for application to normal skin (in other words non-dermatophyte infected skin) as a means of delivering at least one immunosuppressant, or a pharmaceutically acceptable salt, solvate or physiologically functional derivative thereof, for systemic purposes via transdermal application to a patient. In such cases, it can be preferred that a patch forming composition according to the present invention is provided as a metered dose spray formulation which can optimise therapeutic dosing thereby.

The present invention also provides a method for the treatment or prophylaxis of dermatophytosis and related conditions in a patient suffering from, or susceptible to, dernatophytosis and related conditions, which method comprises topically applying to a dermatophyte infected area of skin, or an area of skin susceptible to dermatophyte infection, a therapeutically effective amount of at least one immunosuppressant, or a pharmaceutically acceptable salt, solvate or physiologically functional derivative thereof, provided in a patch, or patch forming composition, substantially as hereinbefore described.

According to the above described method of the present invention, an immunosuppressant, or a pharmaceutically acceptable salt, solvate or physiologically functional derivative thereof, can be topically administered to a dermatophyte infected area of the skin, or an area of the skin susceptible to dermatophyte infection, and as such exert a local therapeutic effect thereto. The treatment regime will be dependent on the patient, and severity of the dermatophyte infection being treated, and will generally be at the discretion of an attendant physician.

The present invention also provides a method for the treatment or prophylaxis of a condition for which systemic administration of at least one immunosuppressant, or a pharmaceutically acceptable salt, solvate or physiologically functional derivative thereof, is indicated, which method comprises topically applying to the skin of a patient a therapeutically effective amount of at least one immunosuppressant, or a pharmaceutically acceptable salt, solvate or physiologically functional derivative thereof, provided in a patch, or patch forming composition, substantially as hereinbefore described, so as to effect transdermal administration and thereby the required systemic treatment of the condition.

There is also provided by the present invention a method of improving the bioavailability in a patient of at least one immunosuppressant, or a pharmaceutically acceptable salt, solvate or physiologically functional derivative thereof, which method comprises administering a therapeutically effective amount of said at least one immunosuppressant, or a pharmaceutically acceptable salt, solvate or physiologically functional derivative thereof, in the form of a patch, or patch forming composition, according to the present invention substantially as hereinbefore described.

According to the present invention, there is further provided at least one immunosuppressant, or a pharmaceutically acceptable salt, solvate or physiologically functional derivative thereof, for use in the manufacture of a patch, or patch forming composition, for the treatment of dermatophytosis and related conditions.

According to the present invention, there is further provided at least one immunosuppressant, or a pharmaceutically acceptable salt, solvate or physiologically functional derivative thereof, for use in the manufacture of a patch, or patch forming composition, for the treatment of a condition for which systemic administration of at least one immunosuppressant, or a pharmaceutically acceptable salt, solvate or physiologically functional derivative thereof, is indicated.

The present invention also provides a process of preparing a patch, or patch forming composition, as described herein, which process comprises providing at least one immunosuppressant, or a pharmaceutically acceptable salt, solvate or physiologically functional derivative thereof, and admixing with a pharmaceutically acceptable carrier or excipient therefor, so as to form a patch, or patch forming composition, according to the present invention substantially as hereinbefore described.

The present invention will now be further illustrated by the following Examples, which do not limit the scope of the invention in any way.

### EXAMPLES

### Example 1

| Sr. No. | Ingredients | % w/w |
|---|---|---|
| 1. | Tacrolimus | 0.1-5% |
| 2. | Lutrol F127 | Qs to 100% |
| 3. | Ethanol | 15-30% |
| 4. | Acetone | 10-40% |
| 5. | Methylene chloride | |

### Example 2

| Sr. No. | Ingredients | % w/w |
|---|---|---|
| 1. | Tacrolimus | 0.1-5% |
| 2. | Lutrol F68 | Qs to 100% |
| 3. | Ethanol | 15-30% |
| 4. | Acetone | 10-40% |
| 5. | Isopropanol | 10-12% |

### Example 3

| Sr. No. | Ingredients | % w/w |
|---|---|---|
| 1. | Tacrolimus | 0.1-5% |
| 2. | Carbomer | Qs to 100% |
| 3. | Ethanol | 15-30% |
| 4. | Acetone | 10-40% |
| 5. | Methylene chloride | 10-12% |

### Example 4

| Sr. No. | Ingredients | % w/w |
|---|---|---|
| 1. | SDZASM-91 | 0.1-5% |
| 2. | Carbomer | Qs to 100% |
| 3. | Ethanol | 15-30% |
| 4. | Acetone | 10-40% |
| 5. | Methylene chloride | 10-12% |

### Example 5

| Sr. No. | Ingredients | % w/w |
|---|---|---|
| 1. | Cyclosporin | 0.1-5% |
| 2. | Lutrol F68 | Qs to 100% |
| 3. | Ethanol | 15-30% |
| 4. | Acetone | 10-40% |
| 5. | Isopropanol | |

### Example 6

| Sr. No. | Ingredients | % w/w |
|---|---|---|
| 1. | Cyclosporin | 0.1-5% |
| 2. | Polyacrylate | 49% |
| 3. | Polyethylene vinyl acetate | 40.1% |
| 4. | Oleic acid | 2.0% |
| 5. | Propylene glycol | 0.8% |
| 6. | Lecithin | 1.2% |
| 7. | Dipropylene glycol | 1.0% |
| 8. | Bentonite | 1.0% |

### Example 7

| Sr. No. | Ingredients | % w/w |
|---|---|---|
| 1. | Tacrolimus | 0.1-5% |
| 2. | Polyacrylate | 49% |
| 3. | Polysiloxane | 40.1% |
| 4. | Oleic acid | 2.0% |
| 5. | Propylene glycol | 0.8% |
| 6. | Lecithin | 1.2% |
| 7. | Dipropylene glycol | 1.0% |
| 8. | Bentonite | 1.0% |

The above compositions were prepared by techniques known in the art so as to provide a patch, or patch forming composition, according to the present invention.

1. A patch, or patch forming composition, comprising said at least one immunosuppressant, or a pharmaceutically acceptable salt, solvate or physiologically functional derivative thereof, together with a pharmaceutically acceptable carrier or excipient therefor, formulated for delivery of said at least one immunosuppressant, or a pharmaceutically acceptable salt, solvate or physiologically functional derivative thereof, to the skin of a patient.

2. A patch, or patch forming composition, according to claim 1, provided in the form of a spray-on patch, a transdermal patch, a physical patch or a controlled release patch.

3. A patch, or patch forming composition, according to claim 1, wherein said at least one immunosuppressant, or a pharmaceutically acceptable salt, solvate or physiologically functional derivative thereof, is selected from the group consisting of tacrolimus, cyclosporine, ascomycin or a pharmaceutically acceptable salt, solvate or physiologically functional derivative thereof.

4. A patch, or patch forming composition, according to claim 1, wherein said at least one immunosuppressant, or a pharmaceutically acceptable salt, solvate or physiologically functional derivative thereof, is present at a concentration in the range of 0.1 wt % to 5 wt % of the total patch composition.

5. A patch, or patch forming composition, according to claim 1, which further comprises a combination of pharmaceutically acceptable polymers included so as to modulate delivery of said at least one immunosuppressant, or a pharmaceutically acceptable salt, solvate or physiologically functional derivative thereof.

6. A patch, or patch forming composition, according to claim 1, which further comprises one or more of the following: at least one film forming agent, at lest one film plasticiser and at least one organic solvent.

7. A patch, or patch forming composition, according to claim 6, wherein said film forming agent suitable for use in said patch, or patch forming composition, is selected from the group consisting of: poloxamers, acrylic acid and its derivatives, polyacrylic acid and its derivatives including polybutylmethacrylate, polymethacrylic acid and polymethacrylate, ascorbyl palmitate, carbomer, carnauba wax, cellulose derivatives including cellulose acetate phthalates, crosscarmellose sodium, hydroxyethyl cellulose, hydroxypropyl cellulose, hydroxypropyl methylcellulose, ethyl cellulose and related compounds, hydroxypropyl methyl cellulose phthalate, hypromellose phthalate, crosspovidone, polyvinyl acetate, cellulose nitrates, polyvinyl alcohol and derivatives thereof, cetyl alcohol and derivatives thereof, microcrystalline wax, polyethylene glycol, polyurethane, polyvinyl acetate phthalate, povidone, silicone rubber and derivatives thereof, shellac and triglyceride derivatives.

8. A patch, or patch forming composition, according to claim 6, wherein said film plasticiser is selected from the group consisting of: polybutylphthalate, benzyl benzoate, dibutyl sebacate, dirrmethyl phthalate, dibutyl phthalate, triacetin, glycol and derivatives thereof, benzyl benzoate, glycerin, mineral oil, lanolin alcohols, petroleum, polyethylene glycol, sorbitol, triethylene citrate, propylene glycol, chlorbutanol, castor oil and gelatin.

9. A patch, or patch forming composition, according to claim 6, wherein said solvent is selected from the group consisting of: alcohols including benzyl alcohol, ethanol, methanol, isopropanol, butanol, isobutanol and diacetone alcohol, chlorinated hydrocarbons including methylene chloride, carbon tetrachloride, trichloroethylene and chlorothene SM, esters including methyl acetate, ethyl acetate, n-propyl acetate, n-butyl acetate, isobutyl acetate, amyl acetate, 2-ethyl hexyl acetate, duPont DBE and Exxate 500, 700, 900, glycol and ether/ester derivatives, ethylene glycol, PM acetate, butyl cellucsolve, carbritol acetate, butyl carbritol acetate, hydrocarbons including toluene and xylene, ketones including acetone, methyl ethyl ketone and methyl butyl ketone, ethers including dimethyl ether, benzyl benzoate, isopropyl myristate, acetonitrile, ethyl oleate, glycerol, glycofurol, propylene glycol, polyethylene glycol, hexane, n-hexane, methylene chloride, methyl chloride, octyl dodecanol, trichloromethane, diethyl phthalate and dibutyl phthalate.

10. A patch, or patch forming composition, according to claim 1, which further comprises one or more preservatives selected from celluloses, including HPMC, HPC, methyl cellulose and ethyl cellulose, alcohols including polyvinyl alcohols and ethanol, spans and tweens, and polysorbates.

11. A patch, or patch forming composition, which further comprises gauze on which the patch, or patch forming composition is embedded or applied.

12. A patch, or patch forming composition, according to claim 11, wherein said gauze is selected from the group consisting of polyurethane films and foams, polyethylene oxide and polyvinyl pyrrolidone hydrogels, hydrocolloids and calcium alginates.

13. A patch, or patch forming composition, according to claim 1, which further comprises a pressure sensitive adhesive.

14. A patch, or patch forming composition, according to claim 1, formulated for use in the treatment of dermatophytosis and related disorders.

15. A patch, or patch forming composition, according to claim 1, fomulated for application to normal skin as a means of delivering said at least one immunosuppressant, or a pharmaceutically acceptable salt, solvate or physiologically functional derivative thereof, for systemic purposes via transdermal application to a patient.

16. A patch forming composition according to claim 15, which comprises a spray-on patch thereby providing a metered dose of said at least one immunosuppressant, or a pharmaceutically acceptable salt, solvate or physiologically functional derivative thereof.

17. A method for the treatment or prophylaxis of dermatophytosis and related conditions in a patient suffering from, or susceptible to, dermatophytosis and related conditions, which method comprises topically applying to a dermatophyte infected area of skin, or an area of skin susceptible to dermatophyte infection, a therapeutically effective amount of at least one immunosuppressant, or a pharmaceutically acceptable salt, solvate or physiologically functional derivative thereof, as provided in a patch, or patch forming composition, according to claim 1.

18. A method for the treatment of prophylaxis of a condition for which systemic administration of at least one immunosuppressant, or a pharmaceutically acceptable salt, solvate or physiologically functional derivative thereof, is indicated, which method comprises topically applying to the skin of a patient a therapeutically effective amount of at least one immunosuppressant, or a pharmaceutically acceptable salt, solvate or physiologically functional derivative thereof, as provided in a patch, or patch forming composition, according to claim 1 so as to effect transdermal administration and thereby the required systemic treatment of said condition.

19. A method of improving the bioavailablity in a patient of at least one immunosuppressant, or a pharmaceutically acceptable salt, solvate or physiologically functional derivative thereof, which method comprises administering a therapeutically effective amount of said at least one immunosuppressant, or a pharmaceutically acceptable salt, solvate or physiologically functional derivative thereof, in the form of a patch, or patch forming composition, according to claim 1.

20. A method for the manufacture of a patch, or patch forming composition, for the treatment of dermatophytosis and related conditions, comprising including at least one immunosuppressant, or a pharmaceutically acceptable salt, solvate or physiologically functional derivative thereof in the patch or patch forming composition.

21. A method for the manufacture of a patch, or patch forming composition, comprising including at least one immunosuppressant, or a pharmaceutically acceptable salt, solvate or physiologically functional derivative thereof, for the treatment of a condition for which systemic administration of at least one immunosuppressant, or a pharmaceutically acceptable salt, solvate of physiologically functional derivative thereof, is indicated in the patch, or patch forming composition.

22. (canceled)

23. A process of preparing a patch, or patch forming composition, according to claim 1, which process comprises providing at least one immunosuppressant, or a pharmaceutically acceptable salt, solvate or physiologically functional derivative thereof, and admixing with a pharmaceutically acceptable carrier or excipient therefore, so as to form said patch, or patch forming composition.
